(19) United States
(12) Patent Application Publication
(10) Pub. No.: US 2017/0045505 A1
(43) Pub. Date: Feb. 16, 2017

(54) ELECTROCHEMICAL DETECTION SYSTEMS AND METHODS USING MODIFIED COATED MULTI-LABELED MAGNETIC BEADS WITH POLYMER BRUSHES

(71) Applicant: SALVE REGINA UNIVERSITY, Newport, RI (US)

(72) Inventor: Bernard S. MUNGE, Middletown, RI (US)

(21) Appl. No.: 15/304,325

(22) PCT Filed: Apr. 14, 2015
(86) PCT No.: PCT/US15/25677

§ 371 Date: Oct. 14, 2016

Related U.S. Application Data
(60) Provisional application No. 61/980,116, filed on Apr. 16, 2014.

(51) Int. Cl. G01N 33/543; G01N 27/327; C12Q 1/68; B01L 3/00; G01N 33/552

(52) U.S. Cl. CPC G01N 33/54326; B01L 3/502761; G01N 33/552; C12Q 1/6834; C12Q 1/6825; G01N 27/3276; B01L 2300/0627; B01L 2200/0647

(57) ABSTRACT

An immunosensor is provided that includes polymer coated particles, wherein the polymer coated particles are labelled with an enzyme and used for at least one of protein biomarker detection and DNA biomarker detection.

### PRIORITY

The present application claims priority to U.S. Provisional Patent Application Ser. No. 61/980,116 filed Apr. 16, 2014, the disclosure of which is hereby incorporated by reference in its entirety.

### GOVERNMENT SUPPORT

The present invention was sponsored, in part, by the United States National Center for Research Resource (NCRR) of the National Institute of Health (NIH) under Grant number P20RR016457. The United States government has certain rights to the present invention.

### BACKGROUND

The invention generally relates to immunosensors and relates in particular to the detection of biomarker proteins for diagnosis and disease monitoring. Highly sensitive and selective immunosensors for early detection of biomarker proteins are critically required for diagnosis and disease monitoring (Ferrari, M., 2005. Nat. Rev. Cancer 5, 161-171; Wulfkuhle, J. D., Liotta, L. A., Petriconie, E. F. 2003. Nat. Rev. Cancer 3, 267-275; Wilson, M. S., Nie, W. Y., 2006. Anal. Chem. 78, 6476-6483; Rusling, J., Munge, B., Sardesai, N., Malhotra, R., Chikkaveeraiah, B., 2012. Nanoscience-Based Electrochemical Sensors and Arrays for Detection of Cancer Biomarker Proteins. In: Crespilho, F. N., (ed.), Nanobioelectrochemistry. Springer Heidelberg, New York, Dordrecht, London, pp. 1-26). Although development of such devices poses a formidable challenge, if realized would allow monitoring of patient's response to therapy, lower treatment costs, stress among patients and families, and provide devices for early cancer screening and point-of-care (POC) diagnosis (Kitano, H., 2002. Science 295, 1662-1664; Srinivas, P. R., Kramer, B. S., Srivastava, S., 2001. Lancet. Oncol. 2, 698-704; and Hood, E., 2003. Environ. Health Perspect. 111, A817).

Interleukin-6 (IL-6), a multi-functional cytokine, involved in inflammatory response is a biomarker protein found at elevated levels in the presence of many different forms of cancers including head and neck squamous cell carcinoma (HNSCC) (Bigbee, W. L., Grandis, J. R., Siegfried, J. M., 2007. Clin. Cancer Research 13, 3107-3108; Chen, Z., Malhotra, P. S., Thomas, G. R., Ondrey, F. G., Duffey, D. C., Smith, C. W., Enamorado, I., Yeh, N. T., Kroog, G. S., Rudy, S., McCullagh, L., Mousa, S., Quezado, M., Herscher, L. L., Waes, C. V. 1999. Clinical Cancer Research 5, 1369-1379; Cohen A. N., Veena, M. S., Srivatsan, E. S., Wang, M. B., 2009. Arch. Otolaryngol Head Neck Surg. 135, 190-197; Richards, B. L., Eisma, R. J., Spiro, J. D., Lindquist, R. L., Kreutzer, D. L. 1997. Am. J. Surg. 174, 507-12). There are approximately 41,000 patients diagnosed with HNSCC each year in the United States and about 8,000 results in death (Siegel, R., Naishadham, D., Jemal, A., 2012. CA: Cancer J. Clin. 62, 10-29). This poor mortality rate is due to the difficulty in early detection and monitoring of specific biomarkers resulting in late diagnosis at advanced metastatic stage (Siegel, R., Naishadham, D., Jemal, A., 2012. CA: Cancer J. Clin. 62, 10-29; Thomas, G. R., Nadiminti, H., Regalado, J., 2005. Int. J. Exp. Pathol. 86, 347-363; and Riedel, F., Zaiss, I., Herzog, D., Götte, K., Naim, R., Hörmann, K., 2005. Anticancer Research 25, 2761-2766). The mean sera concentration of IL-6 in HNSCC patients is ≧20 pg mL⁻¹ compared to ≦13 pg mL⁻¹ in healthy individuals (Riedel, F., Zaiss, I., Herzog, D., Götte, K., Naim, R., Hörmann, K., 2005. Anticancer Research 25, 2761-2766). Such low serum levels present a significant challenge underscoring the need for a ultrasensitive detection method. For reliable clinical applications, changes in both normal and elevated levels of IL-6 need to be accurately measured.

A single biomarker found at an elevated level, however, does not give complete accuracy for a diagnosis. For example, PSA, the most widely used serum biomarker for prostate cancer, has a positive predictive value of about 75% (Lilja, H., Ulmert, D., Vickers, A. J., 2008. Nat. Rev. Cancer 8, 268-278). Recent studies have shown that approximately 100% predictive success may be achieved by measuring 5 to 10 biomarkers of a particular cancer (Hanash, S. M., Pitteri, S. J., Faca, V. M., 2008. Nature 452, 571-579; Stevens, E. V., Liotta, L. A., Kohn, E. C., 2003. Int. J. Gynecol. Cancer 13, 133-139; Wagner, P. D., Verma, M., Srivastava, S., 2004. Ann. N.Y. Acad. Sci. 1022, 9-16; Weston, A. D., Hood, L. J., 2004. Proteome Res. 3, 179-196.). Multi-protein arrays are necessary for point-of-care detection. The ultrasensitive immunosensor development for IL-6 serves as the starting point to the development of the electrochemical immunosensor arrays for many conventional biomarker proteins.

Conventional immunoassay methods, including enzyme-linked immunosorbent assay (ELISA) (Yates, A. M., Elvin, S. J., Williamson, D. E., 1999. J. Immunoassay 20, 31-44; Voller, A., Bartlett, A., Budwell, D. E., 1978. J. Clin. Pathol. 31, 507-520), fluorescence immunoassay (Cesaro-Tadic, S., Dernick, G., Juncker, D., Buurman, G., Kropshofer, H., Michel, B., Fattinger C., Delamarche, E., 2004. Lab on Chip 4, 563-569; Matsuya, T., Tashiro, S., Hoshino, N., Shibata, N., Nagasaki, Y., Kataoka, K., 2003. Anal. Chem. 75, 6124-6132), surface Plasmon resonance (SPR) (Kurita, R., Yokota, Y., Sato, Y., Mizutani, F., Niwa, O., 2006. Anal. Chem. 78, 5525-5531 and Yu, F., Persson, B., Lofas, S., Knoll, W., 2004. Anal. Chem. 76, 6765-70), magnetic bead-based electrochemilumincence (ECL) (Debad, J. B., Glezer, E. N., Leland, J. K., Sigal, G. B., Wholstadter, J., 2004. In: Bard, A. J. (ed.) Electrogenerated Chemiluminescence, Marcel Dekker, N.Y. p. 359), chemiluminescence (Zhan, W., Bard, A. J., 2007. Anal. Chem. 79, 459-463; Kurita, R., Arai, K., Nakamoto, K., Kato, D., Niwa, O., 2010. Anal. Chem. 82, 1692-1697; and Fu, Z., Hao, C., Fei, X., Ju, H. X., 2006. J. Immuno. Methods 312, 61-7), liquid chromatograpy-mass spectrometry (LC-MS) (Hu, S. H., Zhang, S. C., Hu, Z. C., Xing, Z., Zhang, X. R., 2006. Anal. Chem. 79, 923-29; Niederkofler, E. E., Tubbs, K. A., Gruber, K., Nedelkov, D., Kiernan, U. A., Williams, P., Nelson, R. W., 2001. Anal. Chem. 73, 3294-99; and Ishii, A., Seno, H., Watabe-Suzuki, K., Kumazawa, T., Matsushima, H., Suzuki, O., Katsumata, Y., 2000. Anal. Chem. 72, 404-407) and immuno-polymerase chain reaction (PCR) assay (Niemeyer, C. M., Adler, M., Wacker, R., 2007. Nature Protocols 2, 1918-1930) allow reliable protein detection. However, these approaches are yet to meet all requirements for point-of-care diagnosis which require the sensor to be rapid, operationally simple, low cost and highly sensitive to address both levels of the biomarkers in normal and cancer patient serum. More recent approaches involve nanotransistor (Patolsky, F., Zheng, G., Lieber, C. M., 2006. Anal. Chem. 78, 4260-4269), DNA-based biobarcode assays (Giljohann and Mirkin, 2009), immunmobead-based electrochemiluminescence (ECL), chemiluminescent, and fluorescence arrays (Wang, J., 2006. Biosens. Bioelectron. 21, 1887-1892 and Rusling, J. F., Kumar, C. V., Patel, V., Gutkind, J. S., 2010. Analyst 135, 2496-2511). Commercial bead-based assays have DLs of 1-10 pg mL⁻¹ for various analyte proteins (Rusling, J. F., Kumar, C. V., Patel, V., Gutkind, J. S., 2010. Analyst 135, 2496-2511). Although these techniques are viable, they require specially trained personnel and assay kits and instrumental costs are relatively high.

There remains a need therefore, for immunoassay sensors that are fast, operationally simple, low cost and highly sensitive to address both levels of the biomarkers in normal and cancer patient serum.

### BRIEF DESCRIPTION OF THE DRAWINGS

The following description may be further understood with reference to the accompanying drawings in which:

FIG. 1 shows an illustrative diagrammatic view of detection principles of AuNP immunosensors based on PEG protected multi-label detection for protein biomarker detection;

FIG. 2 shows scanning electromicrographs (SEM) images (on the left) of homogeneous dispersions in PBS buffer, pH 7.0 of; (A) control streptavidin modified 1 μm magnetic beads without HRP protein and PEG protection; (C) 1 μm PEG protected HRP coated magnetic beads, (HRP/MB/Ab₂)-PEG bioconjugate and (on the right); (B) and (D) the corresponding intensity particle size distribution obtained from dynamic light scattering (DLS) measurements;

FIG. 3 shows an optimization of; (A) Capture antibody, Ab₁ using (HRP/MB/Ab₂)-PEG at 1 mg mL⁻¹ and (B) Concentration of (HRP/MB/Ab₂)-PEG using optimum [Ab₁] at 10 μg mL⁻¹;

FIG. 4 shows an Amperometric response for GSH-AuNP immunosensor incubated with IL-6 in 10 μL diluted newborn calf serum for 1.25 hr followed by “stealth” polyethylene glycol protected, (HRP/MB/Ab₂)-PEG bioconjugate;

FIG. 5 shows an Amperometric response for AuNP immunosensor incubated with IL-6 labeled on curves or conditioned media containing IL-6 secreted by human squamous cells;

FIG. 6 shows optimization of the immunoreaction time during the sandwich immunocomplex formation step between bound IL-6 sample and HRP/MB/Ab₂-PEG bioconjugate;

FIG. 7 shows results of enzyme activity assay of HRP/MB/Ab₂-PEG bioconjugate activated by H₂O₂ with ABTS as substrate to give colored product with absorbance at 405 nm;

FIG. 8 shows calibration curve of underivatized HRP standards obtained using ABTS turnover rates for determination of the number of HRP molecules per magnetic bead;

FIG. 9 shows a correlation plot of AuNP sensor results for conditioned media samples against results from ELISA determinations for the same representative HNSCC conditioned media samples;

FIG. 10 shows an electrochemical detection system in accordance with an embodiment of the present invention; and

FIG. 11 shows an illustrative diagrammatic view of detection principles of AuNP immunosensors based on PEG protected multi-label detection for DNA biomarker detection.

The drawings are shown for illustrative purposes only.

### DETAILED DESCRIPTION

In accordance with various embodiments, the invention provides ultrasensitive polyethylene glycol (PEG) protected multi-labeled magnetic bead-based immunosensor coupled to GSH-AuNP platform for the electrochemical detection of IL-6 cancer biomarker in serum.

The specially designed PEG protected (HRP/MB/Ab₂)-PEG bioconjugate was used to minimize non-specific binding (NSB) and particle aggregation. The GSH-AuNPs were bioconjugated to the primary antibodies (Ab₁) and used to capture a cancer biomarker, human interleukin-6 (IL-6) in a sandwich electrochemical immunoassay coupled to horseradish peroxidase enzyme labels. The stealth (HRP/MB/Ab₂)-PEG bioconjugate gave extremely low NSB resulting in a remarkable long linear dynamic range, 10 fg mL⁻¹-1000 pg mL⁻¹ and ultralow DL of 10 fg mL⁻¹ (500 aM) for electrochemical detection of IL-6 in 10 μL serum.

The accuracy of the immuonsensor was determined by measuring IL-6 in head and neck squamous cell carcinoma (HNSCC) cell lines with excellent correlation to the standard ELISA method. These (HRP/MB/Ab₂)-PEG based immuonsensors show great promise for the fabrication of ultrasensitive biosensor microarrays for point-of-care cancer diagnosis.

A focus was on using nanostructured electrodes coupled to multi-labeled signal amplification strategies to achieve highly sensitive electrochemical immunosensors. Previously, a non-amplified AuNP immunosensor has been used for the detection of IL-6 with a DL of 10 pg mL⁻¹ in calf serum (Munge, B. S., Krause, C. E., Malhotra, R., Patel, V., Gutkind, J. S., Rusling, J. F., 2009. Electrochem. Commun. 11, 1009-1012). It was reported in Rusling, J., Munge, B., Sardesai, N., Malhotra, R., Chikkaveeraiah, B., 2012. Nanoscience-Based Electrochemical Sensors and Arrays for Detection of Cancer Biomarker Proteins. In: Crespilho, F. N., (ed.), Nanobioelectrochemistry. Springer Heidelberg, New York, Dordrecht, London, pp. 1-26 that a DL of 0.5 pg mL⁻¹ for PSA in serum using ˜1 μm magnetic beads containing ˜7500 HRPs per nanoparticle. Recently, a process using DL of 1 fg mL⁻¹ for IL-8 in serum with 1 μm magnetic beads with ˜500,000 HRP labels/bead has been used (Munge, B. S., Coffey, A. L., Doucette, J. M., Somba, B. K., Malhotra, R., Patel, V., Gutkind, J. S., Rusling, J. F., 2011. Angew. Chem. Int. Ed. 50, 7915-7918). Alternatively, we have used vertically aligned SWNT immunosensors coupled to multi-labeled HRP-multiwall carbon nanotubes (MWNT)-HRP-Ab₂ bioconjugate to obtain a DL of 4 pg mL₋₁ for PSA (Yu, X., Munge, B., Patel, V., Jensen, G., Bhirde, A., Gong, J. D., Kim, S. N., Gillespie, J., Gutkind, J. S., Papadimitrakopoulos, F., Rusling, J. F., 2006. J. Am. Chem. Soc. 128, 11199-11205) in serum. In another strategy we used 0.5 μm multi-labeled polymeric beads, polybeads-HRP-Ab₂ to achieve a DL of 10 pg mL-1 for MMP-3 (Munge, B. S., Fisher, J., Millord, L. N., Krause, C. E., Dowd, R. S., Rusling, J. F., 2010. Analyst 135, 1345-1350) in calf serum.

In accordance with certain embodiments, the invention provides electrochemical immunosensors for detection of both very low and elevated levels of IL-6. The highly sensitive immunosensor is achieved by the use of ˜5 nm glutathione gold nanoparticle (GSH-AuNP) platform, coupled with specially designed polyethylene glycol (PEG) protected ˜1.0 μm magnetic beads conjugated to the detection IL-6 antibody (Ab₂), and thousands of horseradish peroxidase enzyme (HRP) labels (HRP/MB/Ab₂)-PEG via avidin-biotin interaction. PEG polymer brushes are used to minimize NSB and particle aggregation.

This approach provides ˜50,000 HRP labels per binding event and ultra-low NSB levels providing for extremely sensitive monitoring of any changes in serum concentration. The immunosensor is assembled on an electrode with a pyrolytic graphite tip, starting with the platform of GSH-AuNP. The capture antibody (Ab₁) is bound to the platform, followed by the IL-6 antigen. The PEG coated magnetic bead conjugate is added to bind Ab₂ to the antigen. The signal produced through amperometry is proportional to the concentration of IL-6 antigen. The PEG strategy combined with multi-labelling provides high sensitivity and dramatically minimizes NSB and particle aggregation which precludes measurements at higher analyte concentrations.

This is a major challenge in many high sensitivity electrochemical detection methods based on multi-labeled particles for signal amplification. For example, multi-labeled CNT/AuNP-HRP, IL-6 DL, 1.0 pg mL⁻¹, linear range 4-800 pg mL⁻¹ (Wang, G., Huang, H., Zhang, G., Zhang, X., Fang, B., Wang, L., 2011. Langmuir 27, 1224-1231), polybeads with multi-labeled Au nanoparticles, CEA DL, 0.12 pg mL-1, linear range 10³ (Lin, D., Wu, J., Wang, M., Yan, F., Ju, H., 2012. Anal. Chem. 84, 3662-3668), HRP encapsulated Au hollow microsphere, CEA DL, 1.5 pg mL⁻¹, linear range 0.01-200 ng mL⁻¹ (Tang, D., Ren, J., 2008. Anal. Chem. 80, 8064-8070), HRP coated carbon nanosphere AFP, DL 0.02 ng mL⁻¹, linear range 0.5-6 ng mL⁻¹ (Du, D., Zou, Z., Shin, Y., Wang, J., Wu, H., Engelhard, M. H., Liu, J., Aksay, I. A., Lin, Y., 2010. Anal. Chem. 82, 2989-2995), carbon nanotube HRP carriers, IL-6 DL, 0.5 pg mL⁻¹, linear range 0.5-5 pg mL⁻¹ (Malhotra, R., Patel, V., Vaque, P. J., Gutkind, J. S., Rusling, J. F., 2010. Anal. Chem. 82, 3118-3123).

This novel approach utilizing multi-labeled paramagnetic 1.0 μm beads bioconjugate protected with polyethylene glycols polymer brushes for ultra-low NSB, (HRP/MB/Ab₂)-PEG was used to measure both high and ultra-low IL-6 levels with a long linear range of 5-orders of magnitudes from 10 fg mL⁻¹ to 1000 pg mL⁻¹ and a remarkable low detection limit of 10 fg mL⁻¹ in calf serum. This DL represents 1000-fold lower than our previous report on a non-amplified AuNP immunosensor for the detection of IL-6 with a DL of 10 pg mL⁻¹ in calf serum (Munge, B. S., Krause, C. E., Malhotra, R., Patel, V., Gutkind, J. S., Rusling, J. F., 2009. Electrochem. Commun. 11, 1009-1012). The DL is also 50-fold lower than carbon nanotube forest immunosensor coupled to MWCNT-HRP-Ab₂ amplification, DL 0.5 pg mL⁻¹, linear range 0.5-5 pg mL⁻¹ (Malhotra, R., Patel, V., Vaque, P. J., Gutkind, J. S., Rusling, J. F., 2010. Anal. Chem. 82, 3118-3123). The linear range is 200-fold longer than our recent report on electrochemical detection of IL-8 (Munge, B. S., Coffey, A. L., Doucette, J. M., Somba, B. K., Malhotra, R., Patel, V., Gutkind, J. S., Rusling, J. F., 2011. Angew. Chem. Int. Ed. 50, 7915-7918), 1-500 fg mL⁻¹.

The approach of various embodiments of the invention shows great potential for early cancer detection and point-of-care cancer screening. Significantly, systems of the invention are amenable to immunoarray fabrication.

The reagents and materials used in certain embodiments include monoclonal antihuman interleukin-6 (IL-6) antibody, biotinylated antihuman IL-6 antibody, unconjugated antihuman IL-6 for signal amplification protocol, recombinant IL-6 (carrier-free) in calf serum, and streptavidin-horseradish peroxidase (HRP) were from R&D Systems, Inc. (Minneapolis, Minn.). 2.0 kDa Biotin-PEG-NHS (Succinimidyl Carboxy Methyl ester, SCM) was from Creative PEGWorks (Winston Salem, N.C.). HRP (MW 44 000 Da), lyophilized 99% bovine serum albumin (BSA), and Tween-20 were from Sigma Aldrich. Methanol, (99%—spectrophotometric grade), 99.99% Acetic acid (glacial), 99.99% Sodium borohydride (granules), 99.9% Gold (III) chloride trihydrate, PD 10 desalting columns and L-Glutathione (reduced) used in the synthesis of the glutathione protected gold nanoparticle platform were from Sigma Aldrich. Poly(diallyldimethyl ammonium chloride) (PDDA), 20 wt. % in water was also from Sigma Aldrich. Immunoreagents were dissolved in pH 7.2 phosphate saline (PBS) buffer (0.137 M NaCl, 2.7 mM KCl, 8.1 mM Na₂HPO₄, 1.5 mM NaH₂PO₄). 1-(3-(dimethylamino)-propyl)-3-ethylcarbodiimidehydrochloride (EDC) and N-hydroxysulfosuccinimide (NHSS) from Aldrich were dissolved in water immediately before use.

The preparation of the immunosensor of an embodiment involved providing a multilayer composite. In particular, a gold nanoparticle platform was fabricated on the pyrolytic graphite (PG) tip of an electrode using a monolayer of Poly(diallyldimethyl ammonium chloride) (PDDA). Glutathione protected gold nanoparticles (GSH-AuNP) were prepared using a reaction mixture containing, methanol, sodium borohydride and acetic acid, followed by the addition of glutathione and gold chloride to optimally obtain glutathione modified 5 nm gold nanoparticles.

A monolayer of PDDA polyion glue on the PG electrode tip is used to adsorb the GSH-AuNPs via electrostatic layer-by-layer self-assembly. The sandwich immunosensor was fabricated by attaching the capture antibody (Ab₁) on to the GSH-AuNP platform using 30 μL freshly prepared EDC and NHSS in water, washing after 10 minutes, then incubating overnight, for 9 hours, with 20 μL of 10 μg mL⁻¹ primary anti-IL-6-antibody in pH 7.2 PBS buffer. Following the overnight incubation, the immunosensor was washed with 0.05% Tween-20 in PBS buffer for 3 minutes, replacing with new buffer after 1.5 minutes, and then washing with PBS buffer for 3 minutes, for a total of 4 washes. A blocking step included 20 μL of 1% BSA for a 1 h incubation, followed by another wash with 0.05% Tween-20 in PBS buffer then with PBS buffer for 3 minutes each. Washing steps were optimized in previous experiments to minimize non-specific binding (NSB) to achieve the optimum sensitivity (Munge, B. S., Fisher, J., Millord, L. N., Krause, C. E., Dowd, R. S., Rusling, J. F., 2010. Analyst 135, 1345-1350).

For standardization, the immunosensor was incubated with 10 μL of calf serum containing human IL-6 for 1 h 15 min, followed by washing with 0.05% Tween-20 in PBS buffer and PBS buffer for 3 minutes each. Next, 10 μL of 100 μg mL⁻¹ biotinylated detection antibody (Ab₂) in 1% BSA was incubated for 1 h 15 min, followed by washing with 0.05% Tween-20 in PBS buffer and PBS buffer for 3 minutes each. For the amplified system assay, 5% BSA blocking step was used. For moderate sensitivity, the immunosensor was incubated with 10 μL of streptavidin-HRP for 30 min, followed by washing with 0.05% Tween-20 in PBS buffer and PBS buffer for 3 minutes each. For a more sensitive detection, the Ab₂ and HRP incubations were replaced by a PEG protected multi-labeled superparamagnetic bead, (HRP/MB/Ab₂)-PEG bioconjugate (described below).

For detection, the sensor was placed in an electrochemical cell containing 10 mL of pH 7.2 PBS buffer with 1 mM hydroquinone as a mediator. Amperometry was used by rotating the disk at 2000 rpm at −0.3 V vs. SCE, and the injection of 0.4 mM H₂O₂ to generate the electrochemical signal. This same immunoassay was used to detect conditioned media from cell cultures previously described. These samples were also analyzed using a standard human IL-6 Elisa kit.

The preparation of the PEG protected superparamagnetic bioconjugate, HRP/MB/Ab₂-PEG involved mixing and purification. In particular, anti-human IL-6 secondary antibody, Ab₂ was initially bioconjugated to biotin-PEG-NHS (Succinimidyl Carboxy Methyl ester, SCM). 500 μL (0.2 μg mL⁻¹) of IL-6 Ab₂ in PBS buffer was mixed with 5 mg b-PEG-NHS and vortexed gently for 1 hr. The b-PEG/Ab₂ was then purified using PD10 desalting column, followed by determination of the concentration of the antibodies using UV-Vis nanodrop.

At the same time, 200 μL (5 mg mL⁻¹) of streptavidin coated superparamagnetic particles was put in 1.5 microcentrifuge tube, redispersed in 1000 μL PBS buffer, pH 7.4 then washed 3 times by rotating at 1 rotation/sec in a biomagnetic separation platform (MCB 1200, Sigris Research, Inc. CA). The supernatant was magnetically removed, then 0.1 mg mL⁻¹ biotinylated HRP added to a final volume of 500 μL in PBS buffer, pH 7.4 and incubated for 30 min with gentle rotation at 0.5 rotation/sec using the MCB 1200 platform. The supernatant containing unreacted b-HRP was magnetically removed, and the resulting MB/HRP washed 3 times using 1000 μL PBS buffer.

Then the modified secondary antibody, Ab₂/PEG-b was mixed with MB/HRP in 1000 μL PBS buffer and incubated for 30 min by rotating at 0.5 rotations/sec followed by magnetic removal of the supernatants and 3 times washing with PBS buffer. To cap the unreacted biotins on the Ab₂/PEG-b, 0.2 mg of streptavidin was then added to the HRP/MB/Ab₂-PEG-b conjugate followed by 500 μL of PBS buffer with gentle shaking for 10 min. and subsequent 3 times washing in PBS buffer. Further, 5 mg of hydrolyzed b-PEG-NETS was added and incubated with gentle rotations on the MCB platform for 15 min to react with the additional streptavidin, creating more PEG brushes on the bioconjugate.

The supernantant was then magnetically removed from the PEG protected superparamagnetic beads, (HRP/MB/Ab₂)-PEG bioconjugate, redispersed, then followed by a 30 min quenching step in 1000 μL PBS buffer with gentle spinning at 0.5 rotation/sec using the MCB platform. The supernatant was magnetically removed and the bioconjugate washed 3 times in PBS buffer. Finally, the (HRP/MB/Ab₂)-PEG bioconjugate was redispersed in 200 μL (5 mg mL⁻¹, stock concentration) of PBS buffer containing 0.5% tween-20 and stored in the refrigerator at 4° C. and then diluted with PBS+0.1% Tween 20 before use. The bioconjugate was stable for one week.

The quality of the beads was tested using ABTS enzyme activity assay. The wavelength was set at 405 nm, run time at 180 seconds, and cycle time at 2 seconds. This provided a linear increase in the absorbance. Mean diameter and size distribution of the prepared bioconjugate were determined by dynamic light scattering (DLS) using a Malvern Zetasizer Nano ZS while surface characterization was done using JEOL JSM-5900 LV scanning electron microscope (SEM).

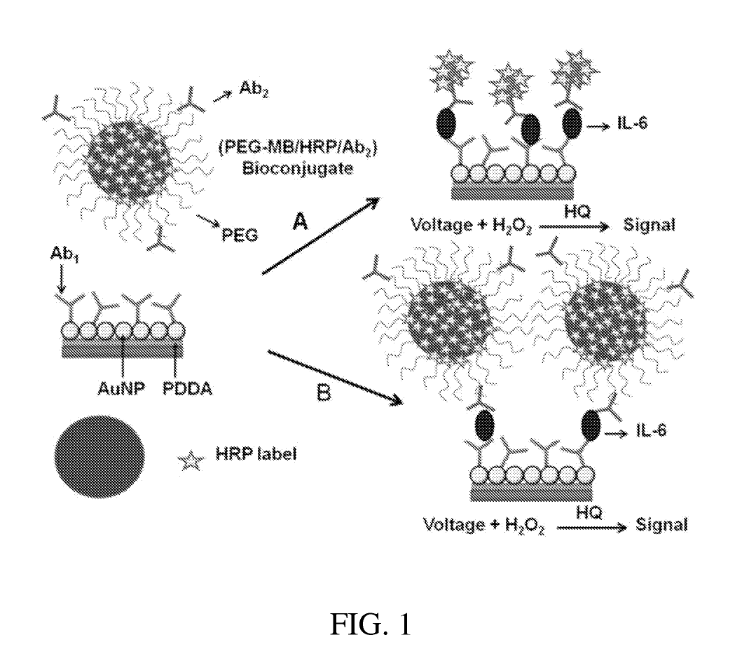

With reference to FIG. 1, the immunosensor strategy of an embodiment of the invention utilized a sensitive Ab₂-biotin-streptavidin-HRP label for moderate sensitivity in each assay. Previously, it has been shown that calibration results for AuNP immunosensors using this 14-16 label system (Munge, B. S., Krause, C. E., Malhotra, R., Patel, V., Gutkind, J. S., Rusling, J. F., 2009. Electrochem. Commun. 11, 1009-1012), indicate a detection limit (DL) of 10 pg mL⁻¹ and sensitivity of 1.6 nA mL cm⁻² (pg of IL-6)⁻¹ as shown at path A in FIG. 1. In accordance with the invention, however, samples with IL-6 levels near or below this detection limit were reanalyzed using an ultrahigh sensitivity polyethylene glycol protected (HRP/MB/Ab₂)-PEG label system as shown at path B in FIG. 1. Calibration curve standards utilized IL-6 dissolved in calf serum which has been shown to approximately match human serum in electrochemical immunoarrays (Yu and Munge et al. 2006). The novel (HRP/MB/Ab₂)-PEG was synthesized and characterized prior to use in the immunosensor device.

As shown in FIG. 1, a sensor of an embodiment of the invention is formed by depositing on an electrode surface, a monolayer of a polymer (PDDA polyion), followed by a layer of gold nanoparticles (AuNP) to which are attached primary antiobodies (Ab₁). Polyethylene glycol coated magnetic beads (1 μm) are provided with secondary antibodies Ab₂ biotin streptavdian HRP labels in solution. As shown at path B, when the biomarker proteins of interest are present, the PEG coated magnetic beads bond to the sensor via antibodies that couple the Ab₁ and Ab₂ antibodies. The resulting composite gives rise to a distinctive electrochemical signal.

FIG. 1 therefore illustrates the detection principles of AuNP immunosensors based on PEG protected multi-label detection in accordance with an embodiment of the present invention. The sensor surface after IL-6 protein capture is shown on the left at the center. Path A on the right shows the immunosensor after treating with biotinylated Ab₂ followed by streptavidin modified HRP resulting in HRP-Ab₂ providing 14-16 label per binding event. Path B on the right shows the immunosensor after treating with stealth polyethylene glycol protected massively labeled (HRP/MB/Ab₂)-PEG particles to obtain amplification by providing ˜50,000 enzyme labels per binding event and ultra-low NSB minimizing particle aggregation.

Techniques of the present invention may generally be applied to any particle carrier such as silica or polymeric particles. The method may also easily be adapted for electrochemical detection of DNA biomarkers via DNA hybridization assays. In this case the primary antibody Ab₁ is replaced with probe 1 with complimentary sequence to one end of the target DNA biomarker and the secondary antibody (Ab₂) is replaced with probe 2 with complementary sequence to the other end of the DNA biomarker as discussed in more detail below.

It has been discovered that the use of PEG protected Bioconjugate reduces aggregation of the magnetic beads. A polyethlylene glycol protected 1 μm magnetic beads bioconjugate containing multiple HRP labels, (HRP/MB/Ab₂)-PEG was synthesized for multi-label amplification (Munge, B. S., Coffey, A. L., Doucette, J. M., Somba, B. K., Malhotra, R., Patel, V., Gutkind, J. S., Rusling, J. F., 2011. Angew. Chem. Int. Ed. 50, 7915-7918; and Wang, J., 2005. Small 1, 1036-43) to enhance sensitivity. The polyethylene glycol outer surface minimizes NSB events (Grainger, D. W., Greef, C. H., Gong, P., Lochhead M. J., 2007. Methods Mol. Biol. 381, 37-57; Masson, J., Battaglia, T. M., Davidson, M. J., Kim, Y., Prakash, A. M. C., Beaudoin, S., Booksh, K. S., 2005 Talanta 67, 918-925; and Reimhult, K., Petersson, K., Krozer, A., 2008. Langmuir 24, 8695-8700) and particle aggregation.

An approach of an embodiment of the invention was to link streptavidin coated 1 μm magnetic beads with biotin-PEG/Ab₂ and biotin-HRP to produce the PEG protected, (HRP/MB/Ab₂)-PEG bioconjugate. The mutilabel particles were used in place of the conventional Ab₂-HRP₍₁₄₋₁₆₎ complex (Munge, B. S., Krause, C. E., Malhotra, R., Patel, V., Gutkind, J. S., Rusling, J. F., 2009. Electrochem. Commun. 11, 1009-1012). This strategy utilizes the strong streptavidin-biotin interaction with a strong binding affinity (Ka of 1015 M⁻¹) (Hoshi, T., Anzai, J., Osa, T., 1995. Anal. Chem. 64, 770-4) to attach the protective PEG layer and the multiple HRP labels on to the paramagnetic beads.

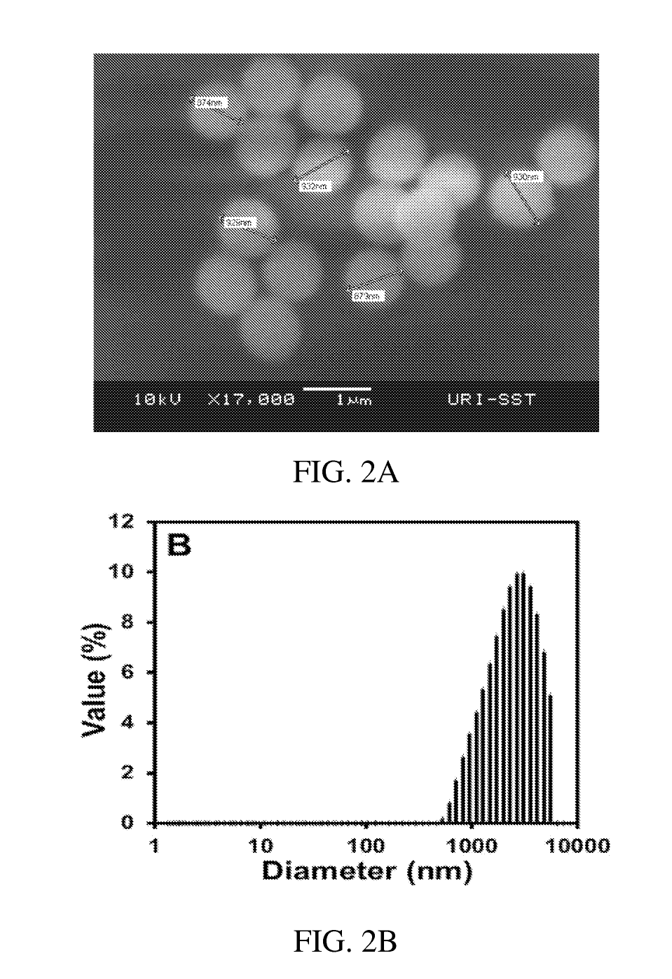

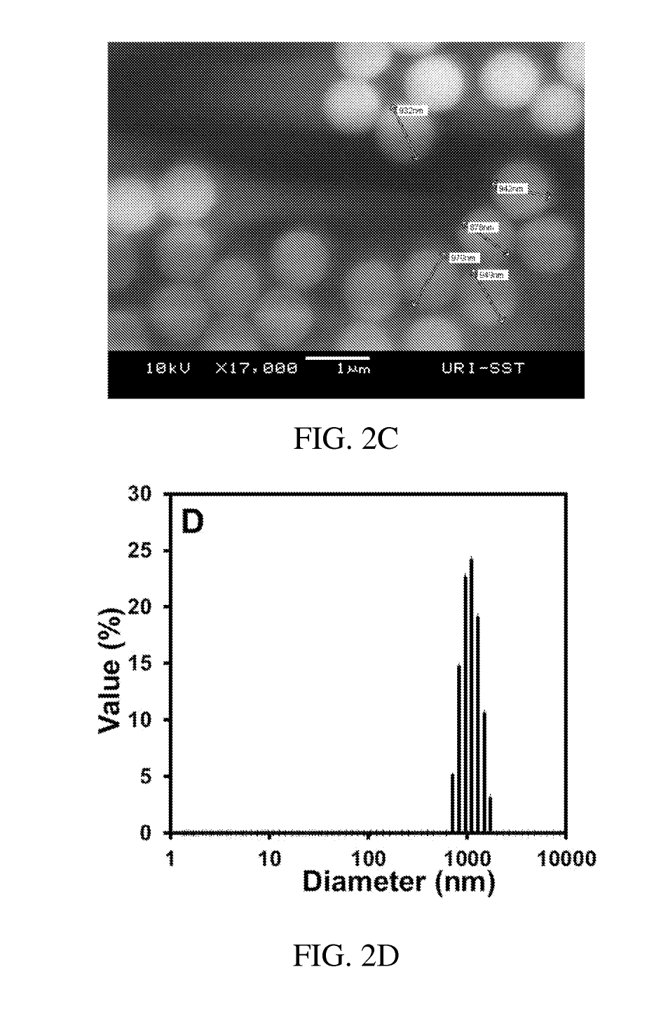

Scanning electron microscope (SEM) images show that the PEG protected, (HRP/MB/Ab₂)-PEG (as shown in FIG. 2C) is well dispersed with no observed clustering compared to the underivatized beads (as shown in FIG. 2A). However, it's difficult to determine aggregation using SEM due to evaporation induced self-assembly (Xu, J., Xia, J., Lin, Z. 2007. Angew. Chem. 119: 1892-1895).

Dynamic light scattering results (as shown in FIG. 2D) show a fairly narrow size distribution of (HRP/MB/Ab₂)-PEG particles with an average particle diameter of 1130±260 nm and a polydispersity index of 0.05 indicative of a monodisperse size distribution (Gaumet, M., Vargas, A., Gurny, R., Delie, F., 2008. Eur. J. Pharmaceutics and Biopharmaceutics 69, 1-9), compared to the control, underivatized MB which show a broad size distribution with an average particle diameter 2302±1540 nm and a polydispersity index of 0.45 suggesting high aggregation level (as shown in FIG. 2B). Broad size distribution was also observed on MB bioconjugate (HRP/MB/Ab₂) without PEG, with average particle diameter of 2526±1525 nm and 0.36 polydispersity index.

FIGS. 2A-2D therefore show SEM images (FIGS. 2A and 2C) of homogeneous dispersions in PBS buffer, pH 7.0 of; control streptavidin modified 1 μm magnetic beads without HRP protein and PEG protection (shown in FIG. 2A); (C) 1 μm PEG protected HRP coated magnetic beads, (HRP/MB/Ab₂)-PEG bioconjugate and the corresponding intensity particle size distribution obtained from dynamic light scattering (DLS) measurements shows dynamic light scattering intensity distribution of MB bioconjugate (HRP/MB/Ab₂) without PEG as a control experiment (shown in FIGS. 2B and 2D). The results show a broad size distribution for the controls with average particle Diameter (nm) Value (%).

FIG. 2B therefore shows dynamic light scattering intensity distribution of MB bioconjugate (HRP/MB/Ab₂) without PEG as a control experiment. In particular, the results show a broad size distribution with average particle diameter of 2536±1525 nm, and polydispersity index of 0.36, suggesting presence of aggregation. NPs aggregate primarily because the attraction between particles is stronger than the attraction for solvent (van Vlerken, L. E., Vyas, T. K., Amiji, M. M., 2007, Pharm. Res. 24, 1405-1414; and Zolnik, B. S., and Sadrieh, N., 2009, Adv. Drug Deliv. Rev. 61, 422-427). NPs with a high surface energy have a greater tendency to aggregate as described by the Derjaguin-Landau-Verwey-Overbeek (DLVO) theory (Guzman, K. Finnigan, M., Banfield, J. 2006, Environ. Sci. Technol., 40, 7688-7693; and Yang, P., Ando, M. Murase, N., 2010, Langmuir 27, 895-901). For spherical NPs, the interaction potential is related to the electrostatic repulsive potential and the van der Waals attraction potential (Yang et al. 2010). PEG decreases the surface energy of NPs and minimizes van der Waals attraction (Jun, Y., Casula, M., Sim, J. H., Kim, S. Y., Cheon, S. Y., Cheon J., Alivisatos, A. P., 2003, J. Am. Chem. Soc. 125, 1581-1585; Förster, S. Antonietti, M., 1998 Adv. Mat. 10, 195-217; and Zhao, W., Brook, M. Li., Y. 2008, ChemBioChem 9, 2363-2371).

Aggregation may also be induced by solvents of high (>100 mM) ionic strength (shielding of solvent from NP), highly concentrated solutions of NPs (less distance between the NPs), time from synthesis, or NP preparations with a very neutral (˜±5 mV) zeta potential (Sze, A., Erickson, D., Ren, L., Li, D. 2003, J. Colloid Interface Sci. 2161, 402-410). PEG decreases the amount of attraction between NPs by increasing the steric distance between them and increasing hydrophilicity via ether repeats forming hydrogen bonds with solvent.

The minimization of aggregation may be attributed to the repulsive electrostatic forces between the PEG polymer chains preventing the magnetic particle bioconjugate from accumulating (Kramer, G., Buchhammer, H. M., Lunkwitz, K., 1998. Colloids Surfaces A: Physicochemical and Engineering Aspects 137, 45-56). The amount of active HRP per unit weight of magnetic beads was determined by reacting (HRP/MB/Ab₂)-PEG dispersion with HRP substrate, 2,2′-azino-bis-(3-ethylbenzthiazoline-6-sulfonic acid) (ABT S) (Matsuda, H., Tanaka, H., Blas, B. L., Nosenas, J. S., Tokawa, T., Ohsawa, S., 1984. Jpn. J. Exp. Med. 54, 131-138) and H₂O₂. This reaction produces a greenish soluble enzymatic reaction product with a characteristic optical density at 405 nm.

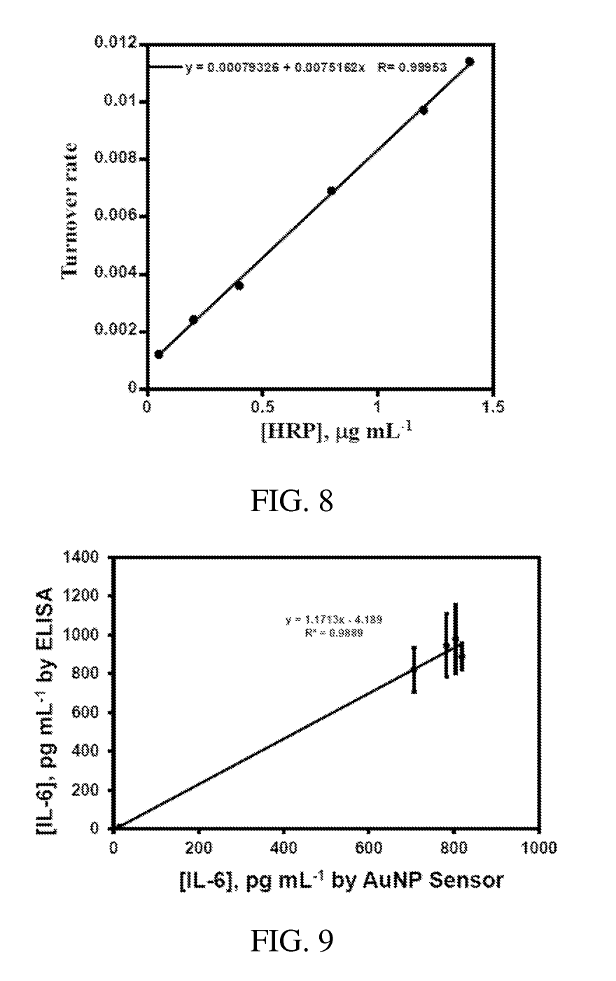

The optical density of the enzyme reaction product increased linearly at 405 nm (as shown in FIG. 7) and the slope was used to estimate (Jensen, G. C., Yu, X., Gong, J. D., Munge, B., Bhirde, A., Kim, S. N., Papadimitrakopoulos, F., Rusling, F. J., 2009. J. Nanosci. Nanotechnol. 9, 249-55) an HRP activity of 48.7 Units mL⁻¹ of undiluted (HRP/MB/Ab₂)-PEG. The background corrected slope was compared to a standard curve constructed with underivatized HRP (as shown in FIG. 8). The concentration of active HRP in the PEG protected stock (HRP/MB/Ab₂)-PEG dispersion was determined in this way to be 58.2 μg mL⁻¹. Considering 0.5 mg of magnetic beads used to prepare the (HRP/MB/Ab₂)-PEG conjugate, we had 3 μmol HRP per mg beads or 15 μmol HRP mL⁻¹ of dispersion. Using the manufacturer's specifications for the magnetic beads with ˜1 μm diameters and a density of 1.592×10¹⁰ beads per mL, the number of active HRP was estimated as 50,000 per bead.

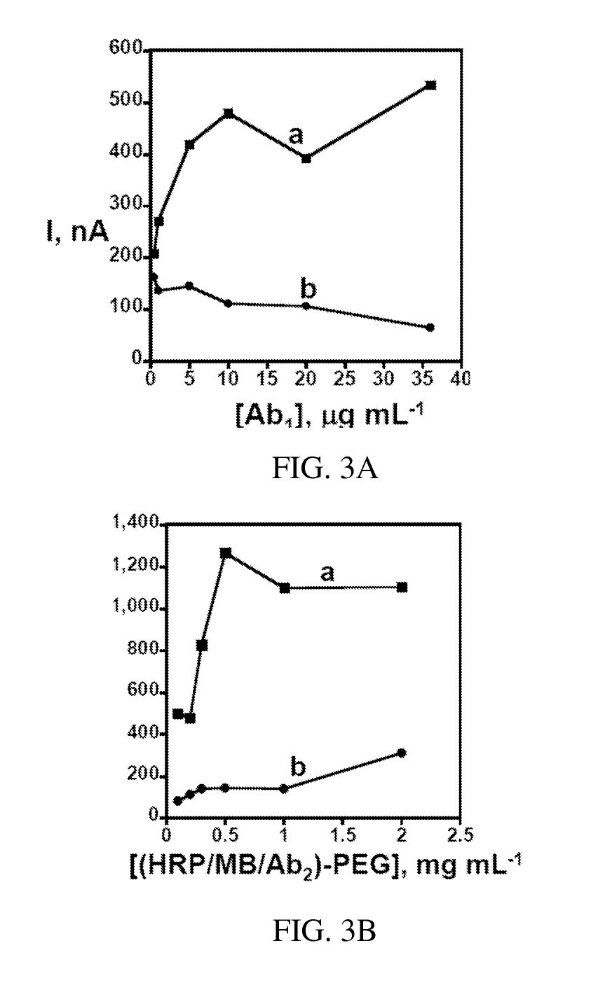

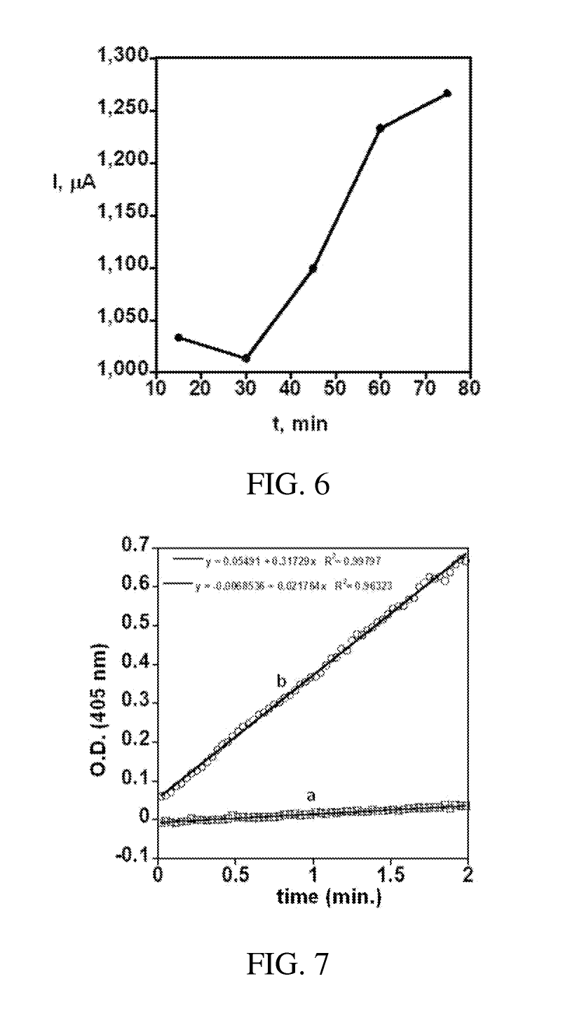

The analytical performance of the immunosensor of this embodiment was optimized while maintaining other parameters constant. In particular, the incubation time between the bound IL-6 and HRP/MB/Ab₂)-PEG bioconjugate was optimized and the best condition determined to be 75 min (see FIG. 6). The analytical performance of the immunosensor was optimized with respective to the concentration of the capture antibody (see FIG. 3A) and concentration of PEG protected multi-labeled magnetic bead bioconjugate, (HRP/MB/Ab₂)-PEG (see FIG. 3B).

The capture antibody concentration was varied while keeping the concentration of the magnetic beads bioconjugate and IL-6 in calf serum at 1 mg mL⁻¹ and 1000 pg mL⁻¹, respectively (see FIG. 3A). Similarly, the magnetic beads bionconjugate concentration was optimized while keeping all the other immuno-reaction parameters constant (see FIG. 3B). The results showed an optimum concentration of 10 μg mL⁻¹ for the capture antibody, Ab₁ and 0.5 mg mL⁻¹ for magnetic beads, (HRP/MB/Ab₂)-PEG bioconjugate. Using these optimized conditions, a calibration curve utilizing this novel polyethylene glycol protected multi-labeled 1 μm superparamagnetic beads, (HRP/MB/Ab₂)-PEG bioconjugate was designed and used to significantly increase the sensitivity of our system.

FIGS. 3A and 3B therefore show optimization of capture antibody, Ab₁ using (HRP/MB/Ab₂)-PEG at 1 mg mL⁻¹ (FIG. 3A) and concentration of (HRP/MB/Ab₂)-PEG using optimum [Ab₁] at 10 μg mL⁻¹. PEG protected multi-labeled, (HRP/MB/Ab₂)-PEG bioconjugate was used to generate the electrochemical signal (FIG. 3B). In particular, FIG. 3A shows incubation with [IL-6] at 1000 pg mL⁻¹ in new born calf serum for 1 h 15 min and FIG. 3B shows a control, indicating full immunoassay with serum containing 0 pg mL⁻¹ IL-6.

A more sensitive system is required in order to detect levels of IL-6 that fall below the 10 pg mL⁻¹ detection limit reported previously (Munge, B. S., Krause, C. E., Malhotra, R., Patel, V., Gutkind, J. S., Rusling, J. F., 2009. Electrochem. Commun. 11, 1009-1012). For clinical applications, an ideal device should be capable of measuring ultra-low and elevated levels of protein cancer biomarkers in serum samples. A major challenge with multi-label amplification using particle carriers is NSB and particle aggregation leading to rapid saturation of the electrode surface at higher concentrations limiting the linear range (Malhotra, R., Patel, V., Vaque, P. J., Gutkind, J. S., Rusling, J. F., 2010. Anal. Chem. 82, 3118-3123; Lin, D., Wu, J., Wang, M., Yan, F., Ju, H., 2012. Anal. Chem. 84, 3662-3668; Tang, D., Ren, J., 2008. Anal. Chem. 80, 8064-8070; Du, D., Zou, Z., Shin, Y., Wang, J., Wu, H., Engelhard, M. H., Liu, J., Aksay, I. A., Lin, Y., 2010. Anal. Chem. 82, 2989-2995). Non-specific binding events usually controls the sensitivity and the detection limits (Ward, A. M., Catto, J. W. F., Hamdy, F. C., 2001. Ann. Clin. Biochem. 38, 633-651; and Wilson, D. S., Nock, S., 2003. Angew. Chem. Int. Ed. 42, 494-500). Competitive binding of bovine serum albumin (BSA) and detergent along with an optimized concentration the primary antibody Ab₁ and (HRP/MB/Ab₂)-PEG (see above) were used to minimize NSB. In addition, novel PEG protected multi-labeled bioconjugate, (HRP/MB/Ab₂)-PEG was designed and used in this ultrahigh sensitivity immunosensor.

The PEG polymer brushes strategy dramatically reduces NSB events and particle aggregation allowing for high sensitivity detection of both ultra-low levels and elevated levels of IL-6 in serum. PEGs are non-branched polymers with high exclusion volumes due to high conformational entropy and therefore repel (bio-) polymers including proteins substantially decreasing NSB of proteins and other macromolecules (Piehler, J., Brecht, A., Valiokas, R., Liedberg, B., Gauglitz, G., 2000. Biosensors & Bioelectronics 15, 473-481).

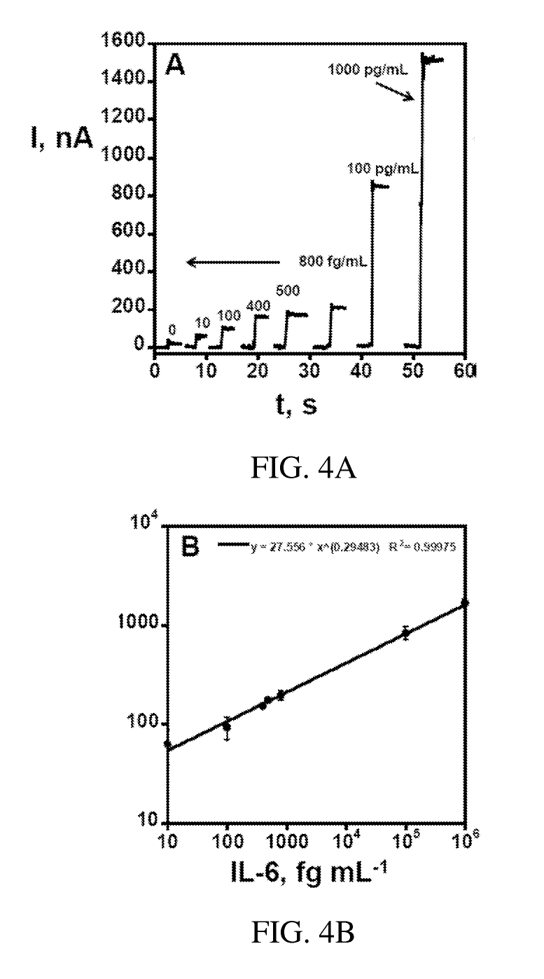

FIG. 4A shows that the steady state sensor current increased with IL-6 concentration in serum over a long linear range of 10 fg mL⁻¹-1000 pg mL⁻¹. A corresponding linear logarithmic calibration curve (as shown in FIG. 4B) is suitable for analysis of samples up to and slightly above 1000 pg mL⁻¹. Sensitivity, determined as the slope of the calibration curve in the linear 10-800 fg mL⁻¹ region, (with reference to FIG. 4B) was 1132 nA mL cm⁻² (pg of IL-6)⁻¹, an increase of ˜708-fold compared with the Ab₂-biotin-streptavidin-HRP system. Good device-to-device reproducibility is illustrated by the small error bars. This approach provided ultra-low detection limit of 10 fg mL⁻¹ (500 aM) as 3 times the average noise plus the zero IL-6 control. The DL is 1000-fold lower than that of the Ab₂-biotin-streptavidin-HRP system. Ultrahigh sensitivity and longer linear range is achieved by using an outer PEG monolayer and highly loaded paramagnetic bead, HRP/MB/Ab₂-PEG bioconjugate which provides about 50,000 HRP/binding event, enabling monitoring of small changes in concentration of IL-6 in a given sample through the amperometric response.

FIG. 4A therefore shows an amperometric response for GSH-AuNP immunosensor incubated with IL-6 in 10 μL diluted newborn calf serum for 1.25 hr followed by stealth polyethylene glycol protected, (HRP/MB/Ab₂)-PEG bioconjugate showing current response at −0.3 V and 2000 rpm after placing electrodes in buffer containing 1 mM Hydroquinone mediator, then injecting H₂O₂ to 0.4 mM to develop the signal. FIG. 4B shows a control with AuNP immunosensor with 0 pg mL⁻¹ IL-6. The corresponding calibration (concentration in fg mL⁻¹) curve of IL-6 immunosensor using HRP/MB/Ab₂-PEG bioconjugate. Errors bars in part B represent device-to-device standard deviations (n=3).

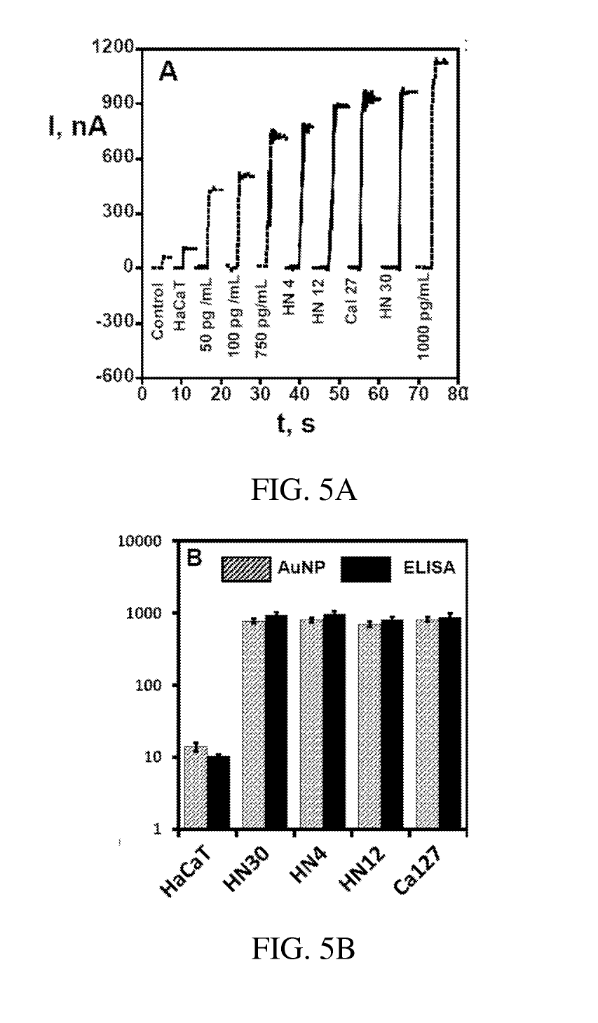

To establish the method's accuracy, the immunosensor was then used to measure secreted levels of IL-6 in in-vitro cell preparations (Malhotra, R., Patel, V., Vaque, P. J., Gutkind, J. S., Rusling, J. F., 2010. Anal. Chem. 82, 3118-3123). IL-6 levels were detected in conditioned media samples from heterogeneous populations of 5 different cell lines in order to test the validity of our immunosensor approach towards IL-6 detection in HNSCC. FIG. 5A shows results along human IL-6 standards in serum at comparable levels. Cell lines HN12, HN30, HN4, Ca127) expressed high levels of IL-6, ranging between 706±59 (RSD 8.3) and 820±60 pg mL⁻¹ (RSD 7.3), while HaCaT demonstrated low levels of the cytokine at 14±0.8 pg mL-1 (RSD 5.7) at 95% confidence level. Replicate unknowns (k=3). To validate the method, samples were also assayed by ELISA, and excellent correlation was found (as shown in FIG. 5B). A correlation plot of the two methods gave a slope near unity at 1.17±0.07 and a correlation coefficient, R2=0.9889 (Fig. S5).

In particular, FIG. 5A shows an amperometric response for AuNP immunosensor incubated with IL-6 labeled on curves or conditioned media containing IL-6 secreted by human squamous cells. Conditioned media samples HaCat, HN4, HN12, Cal27 and HN30 were analyzed using 10 μL of 0.05 mg mL⁻¹ biotinylated secondary antibody (Ab₂) in 0.1% BSA in pH 7.2 PBS buffer and 10 μL of streptavidin modified HRP. FIG. 5A shows current at −0.3 V and 2000 rpm using hydroquinone mediator in PBS buffer, after injecting H₂O₂ to 0.4 mM; and FIG. 5B shows AuNP sensor results for conditioned media shown with results from ELISA for the samples.

The above results demonstrate a sensitive nanostructured immunosensor based on PEG protected multi-label detection for accurate and reproducible determination of human IL-6 cancer biomarker at extremely low 10 fg mL⁻¹ and elevated levels up to 1000 pg mL⁻¹ in calf serum. The PEG coated outer layer on the bioconjugate minimized particle aggregation enabling measurement of high IL-6 concentration resulting in a wide linear dynamic range from low femto gram to a thousand picogram per milliliter of serum solution. This wide range includes IL-6 serum levels in disease-free and cancer patients (Riedel, F., Zaiss, I., Herzog, D., Götte, K., Naim, R., Hörmann, K., 2005. Anticancer Research 25, 2761-2766).

Two sensor approaches were used for moderate and high sensitivity detection. PEG protected (HRP/MB/Ab₂)-PEG bioconjugate with 50,000 HRP labels per bead gave an extremely high sensitivity of 1132 nA mL cm⁻² (pg of IL-6)⁻¹ and ultra-low detection limit of 10 fg mL⁻¹ (FIGS. 4A and 4B). This detection limit is 1000-fold better than the alternative Ab₂-biotin-streptavidin-HRP₍₁₄₋₁₆₎ method and the sensitivity is ˜708-fold better. The detection limit using (HRP/MB/Ab₂)-PEG is also 50-fold better than Ab₂/CNT/HRP method (Malhotra, R., Patel, V., Vaque, P. J., Gutkind, J. S., Rusling, J. F., 2010. Anal. Chem. 82, 3118-3123) and 800-fold better that of ELISA method at 8 pg mL⁻¹ (www.rndsystems.com). The method also has a 100-fold better detection limit than a recently reported dual amplified IL-6 immunosensor based on AuNP-polydopamine combined with CNT/AuNP-HRP multi-labeled detection (Wang, G., Huang, H., Zhang, G., Zhang, X., Fang, B., Wang, L., 2011. Langmuir 27, 1224-1231).

The protocol's detection limit is however similar to the gold discs method (Tang, C. K., Vaze, A., Rusling, J. F., 2012. Lab on a Chip 12, 281-286) but with longer linear range. The PEG protected multi-labeled based immunosensor also showed very good reproducibility demonstrated by small device to device standard deviations (FIGS. 4A and 4B). Furthermore, good accuracy for IL-6 was demonstrated by good correlation of the PEG protected multi-labeled based immunosensor results for IL-6 with ELISA assays for various conditioned media samples. The selectivity of the detection system was established by accurate determination of IL-6 in complex sample matrix containing numerous other proteins.

It has been reported that an ultrasensitive IL-8 immunosensor may be provided based on AuNP coupled with multi-labeled paramagnetic particles, but without an outer PEG coating which gave a short linear range, 1-500 fg mL⁻¹ due to particle clustering leading to early saturation at higher analyte concentration (Munge, B. S., Coffey, A. L., Doucette, J. M., Somba, B. K., Malhotra, R., Patel, V., Gutkind, J. S., Rusling, J. F., 2011. Angew. Chem. Int. Ed. 50, 7915-7918). The present strategy using a PEG protected multi-labeled detection for IL-6 dramatically minimizes NSB and particle aggregation providing a long linear range that can be used for monitoring both ultra-low and elevated levels of cancer biomarkers in serum samples. Such a device may be incorporated in a microfluidic system for simultaneous electrochemical detection of a panel of cancer.

In accordance with various embodiments therefore, the invention provides ultrasensitive, accurate and selective method for electrochemical detection of IL-6 representative of normal patient to high cancer patient levels from a wide range of head and neck cancer cells. The detection limit of 10 fg mL⁻¹ for IL-6 is 1000-fold lower than our previously reported IL-6 immunosensor (Munge, B. S., Krause, C. E., Malhotra, R., Patel, V., Gutkind, J. S., Rusling, J. F., 2009. Electrochem. Commun. 11, 1009-1012), 800-fold lower than that of conventional ELISA (www.rndsystems.com) and 100-fold lower than Quansys Q-plex and bead-based protein assays (www.quansysbio.com). It is also 100-fold lower than the recently reported AuNP-dopamine immunosensor (Wang, G., Huang, H., Zhang, G., Zhang, X., Fang, B., Wang, L., 2011. Langmuir 27, 1224-1231). The PEG protected multi-labeled magnetic bead protocols described can be adapted for measuring other biomarkers and are amenable to array fabrication for the detection of multiple protein biomarkers.

The immunoreaction time was also optimized in certain embodiments. The incubation time effect was examined during the sandwich immunocomplex formation step (between the bound IL-6 sample and (HRP/MB/Ab₂)-PEG bioconjugate) on the performance of the electrochemical immunosensor in the detection of IL-6 at 1000 pg mL⁻¹. FIG. 6 shows that stronger signals were obtained after longer incubation time. The results however, show that after incubation for 75 min there was no further significant increase in current signal. Thus, the optimal incubation time was 75 min.

FIG. 6 shows optimization of the immunoreaction time during the sandwich immunocomplex formation step between bound IL-6 sample and HRP/MB/Ab₂-PEG bioconjugate.

FIG. 7 shows results of enzyme activity assay of HRP/MB/Ab₂-PEG bioconjugate activated by H₂O₂ with ABTS as substrate to give colored product with absorbance at 405 nm.

FIG. 8 shows calibration curve of underivatized HRP standards obtained using ABTS turnover rates for determination of the number of HRP molecules per magnetic bead.

FIG. 9 shows a correlation plot of AuNP sensor results for conditioned media samples against results from ELISA determinations for the same representative HNSCC conditioned media samples. The results show excellent correlation with a slope of 1.17±0.07 close to unity, a y-intercept of −4.18±50 and a coefficient of correlation, R2 of 0.9889.

Generally, an electrochemical detection system in accordance with an embodiment may include a base electrode surface, a polymer layer, and microfluidic channels that are formed of the gold nanoparticles and primary antibodies (Ab₁) discussed above. As also discussed above with reference to FIG. 1, in the presence of the HRP labelled polyethylene glycol coated beads in solution, specimens may be introduced to a microfluidic path at any of multiple input locations. In this way multiple specimens may be tested at the same time, with the electrochemical output current being provided as shown at C₁, C₂ and C₃ respectively.

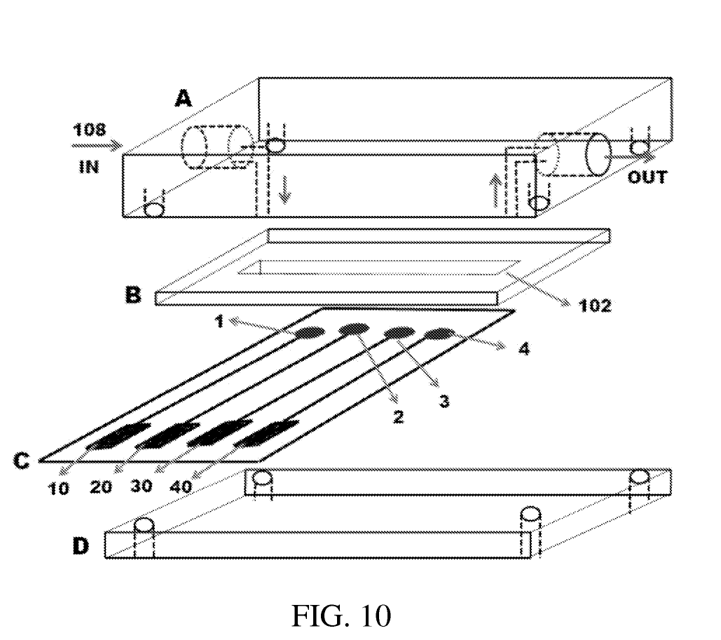

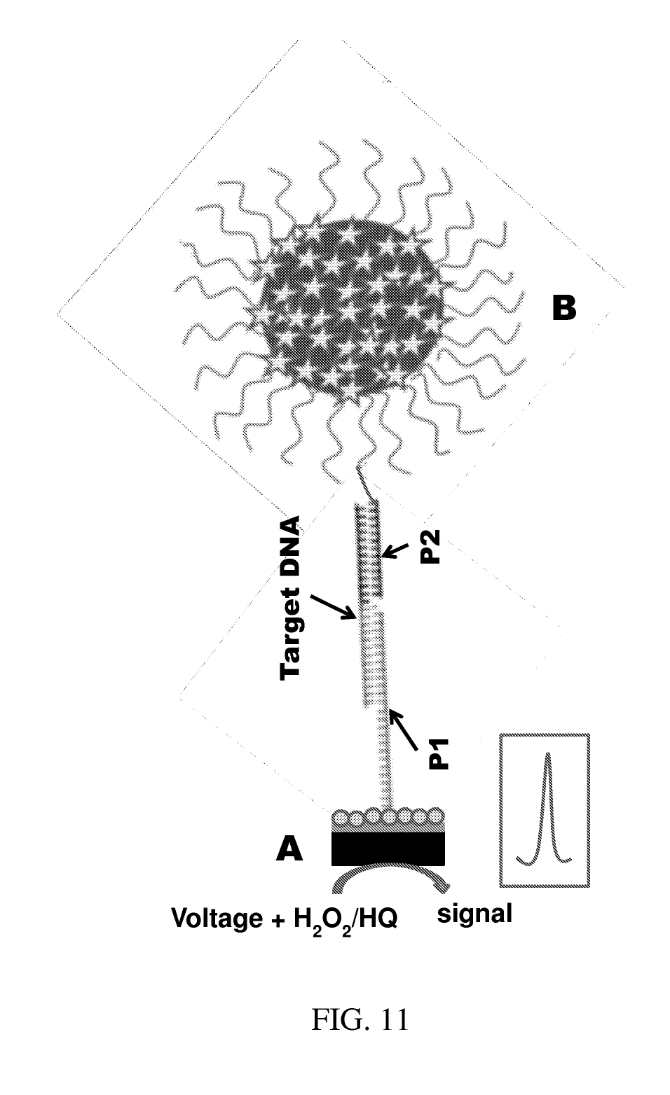

FIG. 10, for example, diagrammatically shows an electrochemical detection system in accordance with an embodiment of the present invention. In particular the electrochemical detection system, 100 includes an electrode array C with detection elements, 1, 2, 3, 4 modified with a polymer layer upon which gold nanoparticles and primary antibodies (Ab₁) specific to different protein biomarkers are attached as discussed above. The electrode array is placed in a microfluidic channel 102. As also discussed above with reference to FIG. 1, in the presence of the HRP labeled polyethylene glycol coated beads HRP/MB/Ab₂)n-PEG bioconjugate mixture upon reaction with specific protein biomarkers in solution, may be introduced to the microfluidic path by flowing through 108 and then allowed to bind to the primary antibody (Ab₁). The electrode array is connected to a multi-potentiostat through electrical contacts 10, 20, 30, 40.

A voltage is applied and H₂O₂ and hydroquinone mediator may be injected through input 108 to generate the electrical current C1, C2, C3 and C4. Polydimethylsiloxane (PDMS) B, may be used to fabricate a microfluidic channel using a suitable mold. This soft PDMS slab with the channel is placed over the electrode array. This assembly is press fitted between two poly(methylmethacrylate) (PMMA) plates A and D to provide a channel. Top PMMA plate features inlet and outlet ports to accept fittings to 0.2 mm PEEK tubing from injector.

FIG. 11 diagrammatically shows an electrochemical DNA detection system via hybridization assay in accordance with a further embodiment of the present invention. In particular the electrochemical detection system, the portion indicated at “A” includes an electrode modified with a polymer layer upon which gold nanoparticles and probe 1 with complementary sequence to one end of a specific target DNA biomarker is attached as discussed above. Then the electrode with attached probe 1 is incubated with sample or standard containing target DNA biomarker to allow hybridization to occur. Then as discussed above the electrode is incubated with the HRP labeled polyethylene glycol coated beads containing probe 2 (with complementary sequence to the other end of the target DNA biomarker) in place of a secondary antibody, Ab₂, HRP/MB/probe-2-PEG as shown at “B” in FIG. 11. Upon hybridization, as discussed, a voltage is applied and H₂O₂ and hydroquinone mediator can be injected through in the cell to generate the electrical current.

Those skilled in the art will appreciate that numerous modifications and variations may be made to the above disclosed embodiments without departing from the spirit and scope of the present invention.

What is claimed is:

1. An immunosensor that includes polymer coated particles, wherein the polymer coated particles are labelled with an enzyme and used for at least one of protein biomarker detection and DNA biomarker detection.

2. The immunosensor as claimed in claim 1, wherein said polymer coated particles are polyethylene glycol coated magnetic beads.

3. The immunosensor as claimed in claim 1, wherein said polymer coated particles include secondary antibodies that are adapted to bind to primary antibodies of a substrate in the presence of protein biomarkers of interest.

4. The immunosensor as claimed in claim 1, wherein said immunosensor provides an electrochemical current signal.

5. The immunosensor as claimed in claim 1, wherein said immunosensor is provided on a microfluidic device.

6. A microfluidic immunosensor system that includes polymer coated particles that are labelled with an enzyme for at least one of protein biomarker detection and DNA biomarker detection.

7. The microfluidic immunosensor system as claimed in claim 6, wherein said polymer coated particles are polyethylene glycol coated magnetic beads.

8. The microfluidic immunosensor system as claimed in claim 6, wherein said polymer coated particles are polyethylene glycol coated silica particles.

9. The microfluidic immunosensor system as claimed in claim 6, wherein said polymer coated particles are polyethylene glycol coated polymer particles.

10. The microfluidic immunosensor system as claimed in claim 6, wherein said polymer coated magnetic particles, silica or polymer particles include secondary antibodies that are adapted to bind to primary antibodies of a substrate in the presence of protein biomarkers of interest.

11. The microfluidic immunosensor system as claimed in claim 6, wherein said microfluidic immunosensor system provides an electrochemical current signal.

12. A method of providing an electrochemical current in an immunosensor system, said method including the steps of providing polymer coated particles that are labelled with an enzyme for at least one of protein biomarker detection and DNA biomarker detection, and permitting the polymer coated magnetic particles to bind to primary antibodies.
